(19) United States
(12) Patent Application Publication
(10) Pub. No.: US 2002/0012920 A1
(43) Pub. Date: Jan. 31, 2002

(54) Method and kit for proteomic identification

(76) Inventors: Kevin Gardner, Montgomery Village, MD (US); Michael Emmert-Buck, Silver Spring, MD (US); Vladimir Knezevic, Gaithersburg, MD (US)

Correspondence Address: LINIAK BERENATO LONGACRE & WHITE, 6550 ROCK SPRING DRIVE, SUITE 240, BETHESDA, MD, 20817, US

(21) Appl. No.: 09/753,574

(22) Filed: Jan. 4, 2001

Related U.S. Application Data
(63) Continuation-in-part of application No. 09/718,990, filed on Nov. 20, 2000.

(51) Int. Cl. C12Q 1/68; G01N 33/53; G01N 33/537; G01N 33/543

(52) U.S. Cl. 435/6; 435/7.92

(57) ABSTRACT

The invention relates to method and kits for facilitating the identification and analysis of proteins and other biological molecules produced by cells and/or tissue, especially human cells and/or tissue. The invention employs a plurality of differentially prepared and/or processed membranes which permit the identification and analysis of proteins, even when present in complex mixtures.

### CROSS-REFERENCE TO RELATED APPLICATION

This application is a continuation-in-part of U.S. patent application Ser. No. 09/718,990, filed on Nov. 20, 2000, herein incorporated by reference in its entirety.

### FIELD OF THE INVENTION

The present invention relates to method and kits for facilitating the identification and analysis of proteins and other biological molecules produced by cells and/or tissue, especially human cells and/or tissue. This invention was made using U.S. Government funds; the U.S. Government may have certain rights in this invention.

### BACKGROUND OF THE INVENTION

Now that the 100,000 or so genes that make up the human genome have been sequenced, a new industry is emerging to ascertain the function of the proteins encoded by these genes, their disease relevance, and the biological molecules that interact with such genes and proteins. This effort, now referred to as “proteomics,” is especially important in efforts to discover new drugs since most new pharmaceutical agents are being designed to interact with enzymes, receptors, and other proteins. Some believe that the 100,000 human genes may turn out to produce up to a million different protein variants. Within the next decade the pharmaceutical industry is expected to identify up to 10,000 proteins against which human therapeutics can be directed.

Additional therapeutics, gene modifiers, expression modifiers, and valuable biomolecules are also expected to be developed or identified through the extension of proteomics to the analysis of non-human animals and plants.

Although there may be up to a million different protein variants in humans, only about 10,000-15,000 proteins are expressed in any particular cell type. Thus, for example, liver cells have essentially the same genome as skin cells taken from the same individual, but the two cell populations express entirely different sets of proteins. It is often desirable, therefore, to profile and compare the patterns of proteins (i.e., the “proteome” of a cell) in different cell populations (e.g. diseased and normal tissue; fetal and mature tissue; human and non-human tissue, etc.) to identify disease targets for drugs.

A number of tools and techniques have been introduced to identify protein expression patterns in biological samples. DNA microarrays such as the GeneChip® system from Affymetrix, Inc. (Santa Clara, Calif.) provide some information on protein expression since mRNA and protein concentrations are sometimes correlated. However, in many cases mRNA and protein levels do not correlate in the cell since many regulatory processes occur after transcription and proteins undergo a myriad of posttranslational modifications including phosphorylation, glycosylation, etc. Thus, a different method of measuring proteins is needed for most proteomic applications.

The most widely used method for identifying and measuring proteins is gel electrophoresis. Electrophoresis is a technique for separating or resolving molecules in a mixture under the influence of an applied electric field based on the difference in their size and charge. Electrophoretic separation of proteins is most commonly performed using porous polyacrylamide gels. During one-dimensional electrophoresis, a mixture of proteins is applied to a gel and exposed to the flow of the electric current. Since smaller proteins migrate faster through the gel than larger ones, separation based on their size is achieved. This unidimensional approach can only generate about 100 distinct protein bands, which is inadequate for many applications since the estimated number of proteins expressed in a typical mammalian cell is between about 10,000-15,000 proteins.

In order to improve the resolving power of electrophoresis gels, a two dimensional gel technique was introduced in the 1970s wherein electrophoresis separation of the proteins based on their size is preceded by charge-based separation. As shown in FIG. 6, isoelectric focusing (IEF) electrophoresis, which separates proteins according to their charge (pH) is run in one direction and mass separation is carried out in a perpendicular direction. Such two-dimensional (2-D) gel electrophoresis (often abbreviated as “2-D PAGE” for two dimensional polyacrylamide gel electrophoresis) has become the backbone of proteomics. The technique is now routinely employed in both pharmaceutical discovery and scientific research settings for characterizing the proteome of different classes of tissues, cells, cell lysates, body fluids or exudates. The end result of 2-D PAGE is the production and separation of various protein “spots” in a two dimension Cartesian plane where the coordinates of each spot are represented by charge and molecular weight. However, the major challenge of 2-D electrophoresis is the identification of the proteins after they have been separated on the gel.

Proteins that have been separated on gels are usually identified, detected and analyzed by one of several different techniques. If the protein spot represents an unknown protein, the most common approach is to physically remove or excise the spot from the gel, digest it with an enzyme, and characterize the protein by mass spectroscopy. A computer generates a plot of protein fragments according to their mass, and this plot serves as a fingerprint that may be used to facilitate the identification of the original protein. As in the analysis of actual fingerprints, the ability of mass spectroscopy to identify a detected protein relies on the prior recovery and analysis of a reference protein whose fragments match those of the detected protein. The identification of a truly new protein by mass spectroscopy remains a significant challenge.

Although mass spectroscopy provides the most incontrovertible data, the method is time consuming, expensive and cannot be accomplished in the absence of expensive core facilities and highly trained personnel. Furthermore, the technique is used only to analyze the proteins that can be stained with a ubiquitous stain such as Coomassie blue. Unfortunately, ubiquitous stains are not sensitive and permit only a small fraction of the proteins in the sample to be visualized. In other words, mass spectroscopy of ubiquitously stained gels does not yield a broad “dynamic range” as it fails to identify certain low abundance—but potentially important—proteins. Among the low abundance proteins that may be left behind by these techniques are tyrosine kinases, cytokines, and transcription factors, which play a key role in many diseases.

An alternative approach to identifying gel separated proteins is immuno-blot analysis, which uses a detectable antibody specific to a protein of interest in lieu of a ubiquitous stain. The proteins are transferred onto a membrane, typically constructed of either nitrocellulose or of polyvinylidene difluoride (PVDF) and antibodies are applied to the membranes. Immuno-blotting is rapid and can be accomplished in less than a day. Also, it is estimated to be about 1000-fold more sensitive than Coomassie blue staining, allowing even low abundance proteins to be identified. It is significantly more specific as well. However, a key limitation of immuno-blotting is that at most only a handful of proteins can be identified on a single blot due to overlapping spots and cross-reactivity with different proteins in the sample. Since the 2-D gel process requires approximately 24 hours to complete, it would be prohibitively time consuming to create enough immuno-blots to identify the large quantity of proteins needed for most proteomics applications.

Thus, there is a clear need to develop techniques that permit large numbers of proteins across a wide dynamic range to be identified in parallel. Information potentially relevant to attempts to address this need can be found in the following references: J. -C. Sanchez et al., “Simultaneous analysis of cyclin and oncogene expression using multiple monoclonal immunoblots,” Electrophoresis 1997, 18 638-641; H. Neumann and S. Mullner, “Two replica blotting methods for fast immunological analysis of common proteins in two-dimensional electrophoresis,” Electrophoresis 1998, 19, 752-757; Manabe, et al, “An Electroblotting Apparatus for Multiple Replica Technique and Identification of Human Serum Proteins on Micro Two-Dimensional Gels,” Annal. Biochem. 1984, 143, 39-45; Legocki and Verma, “Multiple Immunoreplica Technique: Screening for Specific Proteins with a Series of Different Antibodies Using One Polyacrylamide Gel,” Annal. Biochem. 1981, 111, 385-45; and PCT International Publication No. WO045168A1 “Method and kit for identifying or characterizing polypeptides;” all herein incorporated by reference.

However, each of the techniques described in these references suffers from one or more of the following disadvantages: (i) not sensitive enough to detect low abundance proteins, (ii) cannot identify large numbers of proteins in a high-throughput manner, and (iii) requires specialized or sophisticated hardware that leads to loss of protein and a decrease in the resolution the protein spots during the transfer.

For the foregoing reasons, there is a strong need for a method of identifying proteins, and in particular, individual protein components from a complex mixture, and especially those resolved via electrophoretic, chromatographic, or fractionating means, that is sensitive enough to detect proteins in low abundance, yet able to detect large numbers of proteins in a high-throughput manner preferably without requiring expensive and sophisticated laboratory equipment.

### SUMMARY OF THE PRESENT INVENTION

The present invention is directed to a method and kit that satisfies the need for proteomic identification techniques that can identify large numbers of proteins from a biological sample (including low abundance proteins) in a high-throughput manner without expensive or sophisticated instrumentation.

According to one aspect of the method of the present invention, proteins that have been electrophoretically separated on a gel are transferred from the gel onto a stack of membranes constructed and chemically treated to have a high affinity but low capacity for the proteins. This allows the creation multiple replicates of the protein content of the gel. The membranes are then separated and each is incubated with a unique mixture or cocktail of antibodies specific for a particular subset of proteins. In other words, while each membrane has essentially the same pattern of proteins bound to it, different combinations of proteins are made visible on each membrane due to the particular cocktail antibodies selected to corresponds to the particular layer. The antibody cocktails are carefully formulated so that no two antibodies in a cocktail bind overlapping or adjacent protein spots. Thus, proteins spots that are too close together to be discriminated on a single membrane are detected on separate membranes according to the inventive method herein.

The antibodies or other ligands employed are labeled or otherwise detectable using any of a several techniques such as enhanced chemiluminescence (ECL). The membrane blots are scanned or otherwise digitally imaged using one of several commercially available scientific imaging instruments. Software is provided with template images corresponding to each of the membrane images. This allows the identity of the protein in each spot to be confirmed based on its vertical and horizontal position of the spot on the gel. The software also allows the density of each spot to be calculated so as to provide a quantitative read-out as described herein. The software may also have links to a database of images generated from other gels to allow comparisons to be made between different diseased and normal samples.

The present invention is also directed to a kit that includes the a set of the aforementioned membranes, separate vials of antibody cocktails and related detection chemistries, transfer buffer, and instructions or labels that indicate the particular antibody cocktail to be applied to particular membrane. The aforementioned software may also be included in the kit or may be accessible via modem or the Internet.

The method and kit according to the present invention allows up to several thousand discrete protein spots to be identified, annotated, and, at the user's option, compared to the pattern of protein spots generated from other biological samples stored in a database.

A key advantage of the present invention is that it provides a third dimension of protein separation for a biological sample, one additional dimension from the size and charge separations which result from 2-D gels. The layered membranes according to the present invention provide a cost-effective tool for selecting groups of compatible antibodies that can be used to detect subsets of proteins on the same membrane. Once selected these antibody combinations can be packaged in a kit and used repeatedly for the controlled analysis of proteomes displayed on stacked membranes. Since 15-20 replicates or copies can be generated from a single gel and up to ten or more antibodies can be applied to each membrane several thousand different proteins can be identified from a single gel according the method of the present invention in a matter of days.

Since antibodies can be used to detect many post-translational modification of proteins (e.g. phosphorylation) the present invention can be employed to identify protein function as well as structure. In addition to 2-D gels the present invention can be used for one dimensional gels such as the identification of transcription factors separated by a gel-shift assay.

In detail, the invention provides a method of analyzing the proteome of a biological sample comprising the steps of:

(a) separating the protein from another protein present in the sample;

(b) transferring a portion of the separated protein to a plurality of membranes (especially 2, 10, 20 or more) in a stacked configuration;

(c) incubating each of the membranes in the presence of one or more species of predetermined ligand molecules (especially 2, 10, 20 or more) under conditions sufficient to permit binding between the separated protein and a ligand capable of binding to such protein; and

(d) analyzing the proteome by determining the occurrence of binding between the protein and any of the species of predetermined ligand molecules.

The invention additionally provides a method for analyzing the extent of similarity between the proteomes of two or more samples comprising the steps of:

(a) for each such sample, separating a protein of such sample from another protein present in the sample;

(b) for each such sample, transferring a portion of the separated protein to a plurality of membranes (especially 2, 10, 20 or more) in a stacked configuration;

(c) for each such sample, incubating each of the membranes in the presence of one or more species of predetermined ligand molecules (especially 2, 10, 20 or more) under conditions sufficient to permit binding between the separated protein and a ligand capable of binding to such protein; and

(d) analyzing the extent of similarity between the proteomes by comparing the separated proteins of each such sample with the separated proteins of another such sample for the occurrence of binding between the separated protein and any of the species of predetermined ligand molecules.

The invention further provides a method for uniquely visualizing a desired predetermined protein if present in a biological sample, comprising the steps:

(a) separating the proteins present in the sample from one another;

(b) transferring a portion of the separated proteins of the sample to a plurality of membranes (especially 2, 10, 20 or more) in a stacked configuration;

(c) incubating each of the membranes in the presence of one or more species of predetermined ligand molecules (especially 2, 10, 20 or more) under conditions sufficient to permit binding between desired predetermined protein and a ligand capable of binding to such protein; and

(d) visualizing any binding between the protein and any of the species of predetermined ligand molecules.

The invention particularly concerns the embodiments of all such methods wherein the separation of the protein from another protein present in the sample is accomplished by electrophoresis (especially 2-dimensional (2-D) gel electrophoresis).

The invention additionally concerns the embodiments of all such methods wherein the sample is obtained from mammalian cells or tissue, and particularly from human cells or tissue, and the embodiments wherein the mammalian cells or tissue are human cells or tissue and the separated protein is a product of a human gene.

The invention additionally concerns the embodiments of all such methods wherein the transferring of a portion of the separated protein is accomplished by gel transfer.

The invention additionally concerns the embodiments of all such methods wherein at least one of the species of ligand is selected from the group consisting of an antibody, an antibody fragment, a single chain antibody, a receptor protein, a solubilized receptor derivative, a receptor ligands, a metal ion, a virus, a viral protein, an enzyme substrate, a toxin, a toxin candidate, a pharmacological agent, and a pharmacological agent candidate. The invention particularly concerns the embodiments of all such methods wherein at least one of the species of ligand is an antibody or an antibody fragment. The invention further particularly concerns the embodiments of all such methods wherein at least one of the species of ligand is a receptor protein, a solubilized receptor derivative, or a receptor ligand. The invention further particularly concerns the embodiments of all such methods wherein at least one of the species of ligand is a pharmacological agent or pharmacological agent candidate.

The invention additionally concerns the embodiments of all such methods wherein the binding of at least one of the species of ligand is dependent upon the structure of the separated protein. The invention further particularly concerns the embodiments of all such methods wherein the binding of at least one of the species of ligand is dependent or upon the function of the separated protein.

The invention additionally concerns the embodiments of all such methods wherein at least one of the membranes is incubated with more than one species of ligand.

The invention additionally concerns the embodiments of all such methods wherein at least 2 membranes are employed, or the embodiments of all such methods wherein at least 10 membranes are employed, or the embodiments of all such methods wherein at least 20 membranes are employed.

The invention additionally concerns the embodiments of all such methods wherein at least at least 2 ligand species are employed, or the embodiments of all such methods wherein at least 10 ligand species are employed, or the embodiments of all such methods wherein at least 20 ligand species are employed.

The invention further provides a kit for analyzing a proteome comprising:

(a) a plurality of membranes, each having a specific affinity for at least one protein, and

(b) a plurality of reagent species, each adapted to detect one or more specific proteins bound to the membranes.

The invention additionally provides a kit for uniquely visualizing a desired predetermined protein if present in a biological sample, comprising:

(a) a plurality of membranes, each having a specific affinity for at least one protein, and

(b) a plurality of reagent species, each adapted to detect the desired predetermined protein if bound to the membranes.

The invention particularly concerns such kits that optionally include instructions setting forth the particular groups of reagents to be applied to each of the membranes.

The invention further concerns such kits wherein the membranes comprise a porous substrate having a thickness of less than about 30 microns. The invention particularly concerns such a kit wherein the membranes are polycabonate membranes, especially polycabonate membranes coated with a material for increasing the affinity of the membrane to biomolecules, especially nitrocellulose.

The invention particularly concerns such kits wherein the reagent species are selected from the group consisting of an antibody, an antibody fragment, a single chain antibody, a receptor protein, a solubilized receptor derivative, a receptor ligands, a metal ion, a virus, a viral protein, an enzyme substrate, a pharmacological agent, and a pharmacological agent candidate.

With the foregoing and other objects, advantages and features of the invention that will become hereinafter apparent, the nature of the invention may be more clearly understood by reference to the following detailed description of the invention, the appended claims and to the several views illustrated in the drawings.

### BRIEF DESCRIPTION OF THE DRAWINGS

FIG. 1 is a schematic illustration showing the components of a kit according to one embodiment of the present invention.

FIG. 2 is a perspective view of the membrane stack according to the present invention.

FIG. 3 is longitudinal section view of a single membrane according to present invention.

FIG. 4 is a longitudinal section view of a stack of membranes shown with apparatus to transfer proteins from a gel onto the membranes.

FIG. 5 is a schematic illustration showing a hypothetical example illustrating the method of creating the antibody cocktails according to the present invention. The Gel (A) shows proteins as detected by Coomassie Blue staining prior to transfer. Membrane-Laye r#1 (B), Membrane-Layer#2 (C), and Membrane-Layer#3 (C) show proteins detected on membranes with antibodies.

FIG. 6 is a schematic illustration showing the method according to the first embodiment of the present invention.

FIG. 7 is a schematic illustration showing the method according to the second embodiment of the present invention.

FIG. 8 is a schematic illustration showing a comparison between a template image with a sample membrane.

FIG. 9 is a photograph of images of the membranes with biotinylated protein bound to them. Proteins were separated by 1-D PAGE, transferred through the membrane stack and visualized with streptavidin-alkaline phosphatase complex (strep-AP) and enhanced chemiluminescence (ECL)reagent.

FIG. 10 is a photograph of images of the membranes with Rsk and p300 proteins bound to them. Protein separation and blotting was performed as stated in FIG. 7.

FIG. 11 is a photograph of images of the membranes with GAPDH, Alpha-tubulin and Beta-actin bound to them. Proteins were separated by 2-D PAGE, transferred through the membrane stack and visualized with primary-secondary antibody-alkaline phosphatase complex and ECL reagent.

FIG. 12 is a photograph of images of the membranes with protein or DNA attached to them and a diagram that explains the relationship between different protein-DNA complexes and their position in the gel.

### DESCRIPTION OF THE PREFERRED EMBODIMENTS

### Definitions

“Biological sample” means any solid or fluid sample obtained from, excreted by or secreted by a living organism (including microorganisms, plants, animals, and humans).

“Affinity” means the chemical attraction or force between molecules.

“Capacity” means the ability to receive, hold, or absorb proteins from the sample.

“Detector” means a molecule, such as an antibody or DNA probe, that is free in solution (i.e. not anchored to a membrane) and has an affinity for one of the sample components.

“Antibody cocktails” means mixtures of between two to about 100 different detector antibodies.

“Identical” means having substantially the same affinity for proteins.

“Membrane” means a thin sheet of natural or synthetic material that is porous or otherwise at least partially permeable to proteins.

“Stack” means adjacent membranes, whether oriented horizontally, vertically, at an angle, or in some other direction. The membranes may be touching or spaced.

“Proteomics” means the identification or analysis of a proteome. A proteome is the group of proteins expressed and/or present in a biological sample.

“Counter-ligand staining” is intended to refer to any detection technique that detects the presence of ligand that is not bound to a protein of the biological sample, and thus reveals (as, for example, by an absence of staining, etc.) the presence of ligand that is bound to a protein of the biological sample

According to the method of the present invention, proteins that have been electrophoretically separated on a gel, or via chromatography, etc. are transferred from the gel onto a stack of membranes. Preferably, most, and more preferably, all of the membranes will be constructed and chemically treated to have a high affinity but low capacity for proteins. Suitable membranes and methods for their construction and preparation are described below and in U.S. patent application Ser. No. 09/718,990, herein incorporated by reference. The use of such membranes allows the creation of multiple replicates of the protein content of the gel. The membranes are then incubated with a unique ligand species or mixture or cocktail of ligand species. The membranes are separated one from another prior to such incubation. Such ligands are preferably antibodies (especially monoclonal antibodies), antibody fragments (e.g., FAB, F(AB)₂, single chain antibodies, receptor proteins, solubilized receptor derivatives, receptor ligands, metal ions (particularly paramagnetic or radioactive ions), viruses, viral proteins (e.g., human rhinovirus or proteins thereof that bind to ICAM-1, or HIV or proteins thereof that bind to CD44), enzyme substrates, toxins, toxin candidates, pharmacological agents, pharmacological agent candidates, other small molecules that bind to specific proteins, etc. While each membrane has essentially the same pattern of proteins bound to it, different combinations of such proteins are detected on each membrane due to the particular ligand or cocktail of ligands selected to corresponds to the particular layer.

The nature of the species of ligand(s) in the cocktail provided to the membrane determines the nature of information that can be obtained from that membrane. For example, by incubating a membrane with an antibody or antibody fragment, one is able to identify the presence or absence of protein molecules of the sample that bind to such molecules. In this way, for example, a membrane could be incubated with an antibody that specifically binds a protein kinase, in order to determine whether a particular protein is a protein kinase, or possesses an epitope that mimics that of a protein kinase. Similarly, by employing as the ligand, a cellular receptor protein, solubilized receptor derivative, or receptor ligand, the membrane would enable one to identify whether a particular protein was a receptor or receptor ligand. Since viruses and other pathogens are capable of binding to cellular receptor proteins, a cocktail containing a virus or viral protein could be employed in the same manner as a receptor ligand to identify whether a particular protein was a cellular receptor or receptor ligand. In an alternative embodiment, the cocktail could comprise one or more pharmacological agents to identify proteins that interact with such agents. Likewise, pharmacological agent candidates could be incubated with the membranes, thereby revealing the ability of such candidate molecules to bind to specific proteins. For example, an acetylcholinesterase inhibitor or a monoamine oxidase inhibitor (MAOI) could be incubated with a membrane to identify proteins that bind the inhibitor and which thus might be additional therapeutic targets of the inhibitor. Likewise, a compound suspected of possessing therapeutic potential could be incubated with a membrane to reveal whether it binds to proteins expressed, for example, in the liver or kidney, thereby revealing its potential to treat diseases affecting these organs. The methods and kits of the present invention permit the further analysis of such binding to determine, for example, whether such proteins are expressed in other organs and tissues (e.g., the brain).

In one embodiment, a membrane will be incubated in the presence of a single ligand, or a cocktail of different ligands of the same class of ligands (e.g., antibodies, receptors, etc.). Alternatively, a membrane may be incubated with different classes of ligands. For example, a membrane that is incubated with antibodies that bind protein kinases and with a therapeutic candidate, can be employed to reveal therapeutic candidates that bind to protein kinases. Where mixtures or cocktails of ligands are employed, the cocktails are preferably formulated so that no two ligands bind overlapping or adjacent protein spots. Thus, proteins spots that are too close together to be discriminated on a single membrane may be detected on separate membranes according to the inventive method described herein.

In an alternative embodiment, the ligand is permitted to bind to proteins of the sample prior to the transfer to a membrane. Thus, the ligand is provided to a living or deceased animal, to a tissue or cell preparation, or to a tissue or cell extract, prior to the fractionation or separation of protein. The proteins are then transferred to membranes and the proteins and ligand are visualized. In this embodiment, one can detect whether binding between a ligand and a protein of the sample and occurs in situ, and/or under physiological conditions. Optionally, one can incubate the membranes in the presence of additional ligand (which may be the same or different from the initially employed ligand) in order to detect competition between or among ligands for binding sites, to evaluate the avidity of binding, etc.

The ligands employed are preferably labeled or otherwise made detectable using any of several techniques, such as enhanced chemiluminescence (ECL), fluorescence, counter-ligand staining, radioactivity, paramagnetism, enzymatic activity, differential staining, protein assays involving nucleic acid amplification, etc. The membrane blots are preferably scanned, and more preferably, digitally imaged, to permit their storage, transmission, and reference. Such scanning and/or digitalization may be accomplished using any of several commercially available scientific imaging instruments (see, e.g., Patton, W. F. et al., Electrophoresis (1993) 14:650-658; Tietz, D. et al., Electrophoresis (1991) 12:46-54; Spragg, S. P. et al., Anal Biochem. (1983) 129:255-268; Garrison, J. C. et al., J Biol Chem. (1982) 257:13144-13149; all herein incorporated by reference). In a preferred embodiment, software is provided with template images corresponding to each of the membrane images. Such software allows the identity of the protein in each spot to be confirmed based upon the vertical and horizontal position of the protein's spot on the gel. Such software also preferably allows the density of each spot to be calculated so as to provide a quantitative, or semi-quantitative read-out as described herein. Such software may also have links to a database of images generated from other gels to allow comparisons to be made between different diseased and normal samples, or links to images or data (structure, sequence, function, etc.).

The present invention is also directed to a kit that preferably includes one or a set of more than one of the aforementioned membranes, and one or more vials of ligand cocktail (which each may contain one or more ligands, as discussed above). Such kits may additionally contain reagents for effecting the detection of ligand-protein binding, buffer, and/or instructions or labels that indicate the particular cocktail to be applied to a particular membrane. The aforementioned software may also be included in the kit or may be accessible via modem, the Internet, by mail, or by other means.

The method and kit according to the present invention allows up to several thousand discrete protein spots to be identified, annotated, and, at the user's option, compared to the pattern of protein spots generated from other biological samples stored in a database.

A key advantage of the present invention is that it provides a third dimension of protein separation for a biological sample, one additional dimension from the size and charge separations obtainable from 2-D gels. The layered membranes according to the present invention provide a cost-effective tool for selecting groups of compatible antibodies that can be used to detect subsets of proteins on the same membrane. Once selected these ligand combinations can be packaged in a kit and used repeatedly for the controlled analysis of proteomes displayed on stacked membranes. Since 15-20 replicates or copies can be generated from a single gel and ten or more ligands can be applied to each membrane several thousand different proteins can be identified from a single gel according the method of the present invention.

Since ligands can be used to detect many post-translational modification of proteins (e.g. phosphorylation) the present invention can be employed to identify protein function as well as structure.

Although the invention has been described with respect to 2-D gels, it may be employed with one dimensional gels (e.g., as for the identification of transcription factors separated by a gel-shift assay), or proteins may be separated from other proteins of a sample, by other means, as by chromatography.

In addition to their use in identifying the proteins of the proteome, the methods and kits of the present invention can be used to measure the concentration of a protein (either in absolute terms, or relative to the concentration of another protein). Likewise, the methods and kits of the present invention can be used to measure the distribution of variants of a protein. The methods and kits of the present invention may be used to identify proteins that are analogous in structure or function to identified human proteins, or to identify plant clones or transgenic animals that express a particular protein or protein variant (preferably linked to, or associated with, a trait or phenotype).

With the foregoing and other objects, advantages and features of the invention that will become hereinafter apparent, the nature of the invention may be more clearly understood by reference to the following detailed description of certain preferred embodiments of the invention, and to the several views illustrated in the drawings.

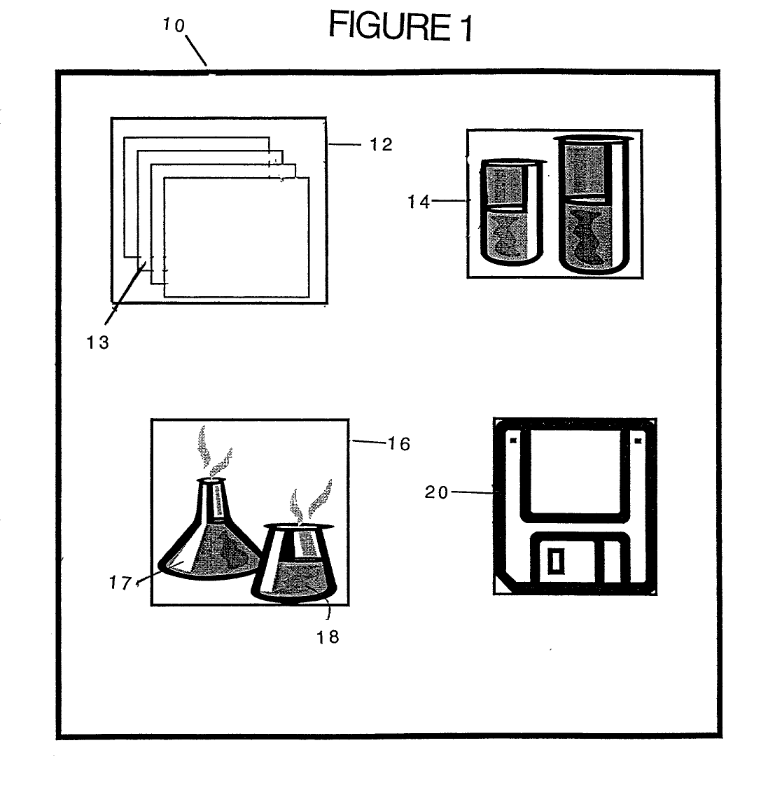

In one embodiment, the present invention is directed to a method and a kit 10 for identifying (i.e. detecting, annotating, and/or characterizing) groups of proteins 11 that have been separated by gel electrophoresis. As illustrated in FIG. 1, in a preferred embodiment of the present invention kit 10 generally comprises the following components: (i) a stack of membranes 12 upon which the proteins are transferred, (ii) primary antibody cocktails 14 one for each of the membranes 12, and (iii) other reagents 16 including protein transfer buffer 17 and antibody detection chemistries 18. The kit may also include software 20 that allows the user to analyze and manipulate the images produced so as to yield a “proteomic image” of the biological sample being tested and compare it to proteomic images from other samples in a database. Alternatively the software may be acquired or accessed independent of the kit.

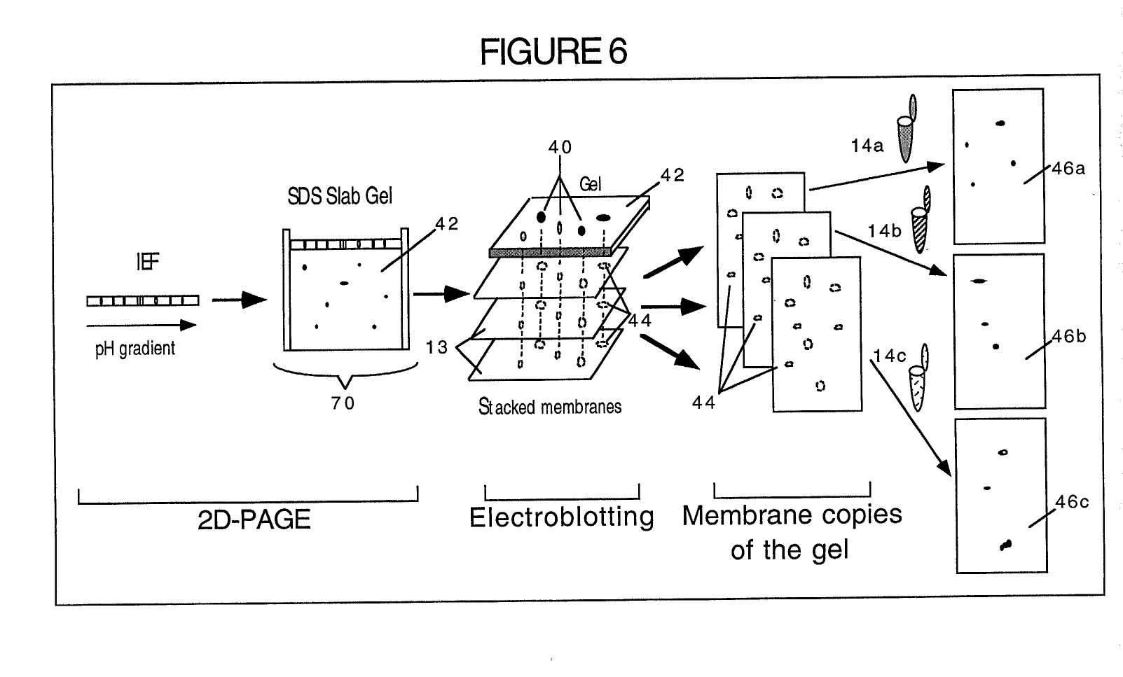

According to the method of a first such embodiment of the present invention (FIG. 6), proteins 40 that have been electrophoretically separated on gel 42 are transferred from the gel through membrane stack 12. This allows the creation of multiple replicate blots 44 of the protein content of the gel. The membranes are then separated and each is incubated with one of the unique cocktails 14(a-c) of ligands, e.g., antibodies. The antibodies employed are labeled or otherwise detectable using any of a several techniques such as enhanced chemiluminescence (ECL). This produces unique spot patterns 46(a-c) on each of the membranes. The membranes with unique spot patterns 46 are then scanned or digitally imaged using an imaging instrument (not shown) so that the density of the spot may be calculated, compared to other samples, and displayed on a computer using software 20, as described herein. An exemplary method and kit that may be employed in accordance with such first embodiment of the present invention are described below in more detail.

### Membrane Construction & Coating

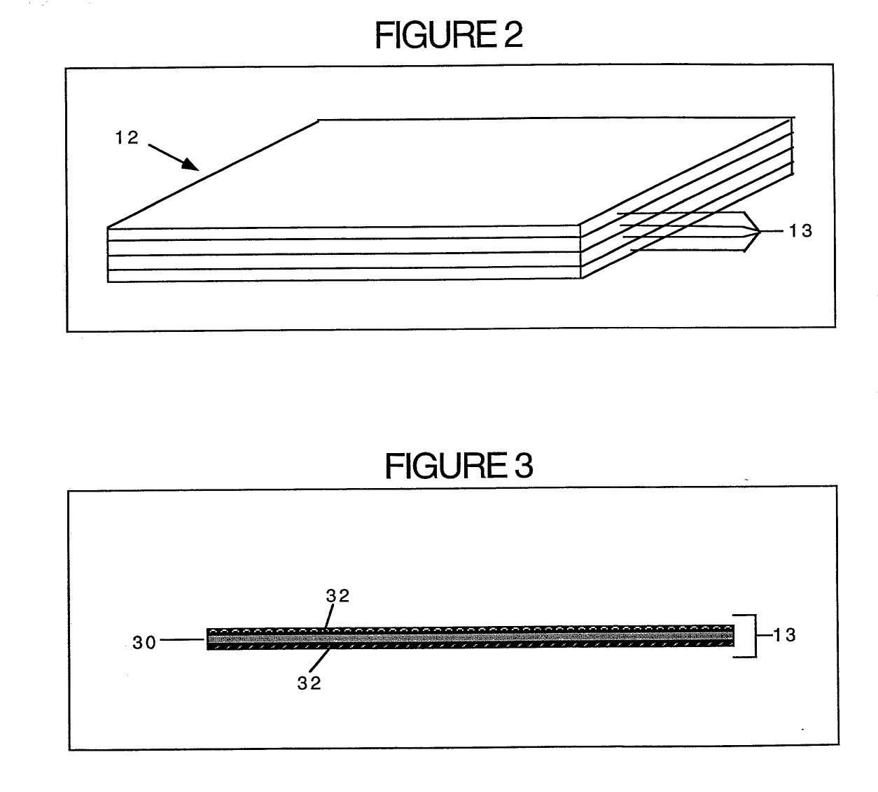

With reference to FIG. 2, membrane stack 12 comprises a plurality of membranes 13 adapted to be removably stacked atop one another as shown. The area of protein separation resulting from most 2-D gels is preferably between about 10×10 cms to 20×20 cms so that the size of membranes 13 varies accordingly.

Membranes 13 are preferably constructed in the manner disclosed in U.S. patent application Ser. No. 09/718,990, filed on Nov. 20, 2000, which is incorporated by reference herein in its entirety. As shown in FIG. 3, membranes 13 are constructed of a porous substrate 30 coated with a material 32 which increases the affinity of the membrane to all of the proteins being transferred. Substrate 30 is preferably constructed of polycarbonate or a similar polymeric material that maintains sufficient structural integrity despite being made porous and very thin. However, in lieu of polycarbonate the substrate 30 may be alternatively constructed of cellulose derivatives such as cellulose acetate, as well as polyolefins, (e.g. polyethylene, polypropylene, etc.), gels, or other porous materials.

It is a particular feature of this embodiment of the present invention that membranes 13 have a high affinity for proteins but have a low capacity for retaining such molecules. This feature permits the molecules to pass through the membrane stack with only a limited number being trapped on each of the successive layers thereby allowing multiple replicate copies to be generated. In other words, the low capacity allows the creation of multiple replicates as only a limited quantity of the proteins are trapped on each layer. More specifically, the affinity and capacity of membrane 13 should be such that when at least 5 and preferably more than 10 membranes are stacked and applied to a gel according to the method of the present invention most of the proteins of interest can be detected on any and all of the membranes including those positioned furthest from the sample. If a membrane were used that had a high binding capacity for proteins—such as the transfer membranes used with conventional gel blotting, multiple replicas could not be made in this manner unless the binding capacity of the membrane was overwhelmed by the amount of protein applied to the membrane.

To ensure that the binding capacity of membrane 13 is sufficiently low to prevent trapping of too much of the sample, the thickness of substrate 30 should preferably be less than about 30 microns, preferably between 4-20 microns and most preferably between about 8 to 10 microns. The pore size of the substrate should preferably be between about 0.1 to 5.0 microns, most preferably about 0.4 microns. Another advantage of using such a thin membrane is that is lessens the phenomenon of lateral diffusion. The thicker the overall stack, the wider the diffusion of proteins moving through the stack.

Substrate 30 includes a coating 32 on its upper and lower surfaces to increase specific binding of the proteins or other targeted proteins. Coating 32 is preferably nitrocellulose but other materials such as poly-L-lysine may also be employed. Before being applied to substrate 30, the nitrocellulose is dissolved in methanol or other appropriate solvent in concentration from 0.1%-1.0%. The membranes are immersed in this solution as described more fully in the Examples, below. In lieu of coating 32, nitrocellulose or other materials with an affinity for proteins can be mixed with the polycarbonate before the substrate is formed thereby providing an uncoated substrate having all of the desired characteristics of the membrane. Alternative coating methods known in the art may be used in lieu of dip coating including lamination. In all instances it should be understood that only one surface—the surface that faces the sample—may be coated instead of both.

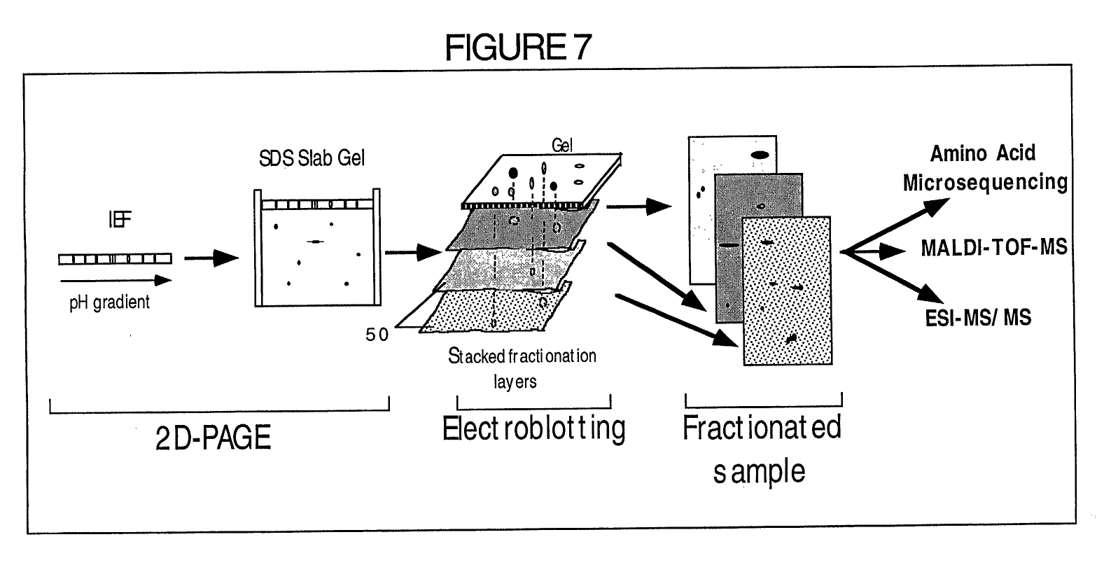

In a second embodiment of the invention (illustrated in FIG. 7), each of the membranes 50 comprises a unique ligand coating that selectively binds to proteins in the biological sample based on a particular characteristic of the protein chemistry (e.g. hydrophobicity, carbohydrate content, etc.) As a result, the membranes 50 function to fractionate the proteins rather than replicate them as with membranes 13 in the first embodiment. The coating could be made in many different ways so that each membrane binds a selective subset of the total protein content in the sample. For example, carbon chains of increasing length, starting with a small carbon molecule can be used in the coating. As the number of carbons increases the ability to bind to proteins increases. Thus, for example, the first layer may have a six carbon-chain coating and will only bind to the most hydrophobic proteins in the sample, the remaining proteins will pass through to the next layer; the second layer has an eight-carbon chain and will pull out slightly less hydrophobic proteins while the remaining proteins pass through; the third layer has a ten carbon-chain, etc.

Thus, with the second embodiment of the invention, each of the membranes will bind to a different group of proteins essentially permitting “3-D gel electrophoresis” by allowing proteins to be separated into three dimensions: in the X and Y dimensions by charge and mass, and then in the Z dimension by an additional chemical characteristic. The proteins on the membranes according to the second embodiment can be visualized by the immuno-staining and imaging methods set forth below. They may also be advantageously analyzed by mass spectrometry either without additional cleavage or after such cleavage (see, PCT WO00/045168), or by other means.

The methods and kits of the present invention facilitate such analysis because the stratification by the different membranes helps to expose moderate and low abundance protein spots that would otherwise be undetectable on standard 2-D gels. The more spots that are available for analysis, the more data can be generated by mass spectroscopy or by such other approaches.

### Detection Chemistries & Cocktails

After proteins 40 have been transferred through stack 12 the individual membranes layers 13 are separated and each is incubated in a separate antibody cocktail 14. A key advantage of creating multiple replicate gel blots according to the present invention is that far more antibodies can be usefully employed as detectors than if all of the antibodies had to be crowded onto a single gel blot.

An exemplary process for designing the ligand cocktails of the present invention—and for determining which proteins will be identified on each membrane layer—is provided below. First the panel of proteins of interest is selected. These can be randomly selected proteins and/or proteins that are not directly related to one another or may be groups of known proteins previously implicated to play a role in one or more particular cellular phenomena (e.g. apoptosis, cell cycle progression) or a particular disease (e.g. prostate cancer specific antigen, PSA). These should be proteins that have been characterized by sequence or coordinates on 2-D gels or for which ligands have been or could be generated. Data bases of annotated 2-D gels include the Quest Protein Database Center (http://siva.cshl.org), the Swiss 2-D PAGE database (http://expasy.cbr.nrc.ca/ch2d), Appel, R. D. et al. “SWISS-2DPAGE: a database of two-dimensional gel electrophoresis images,” Electrophoresis. 1993 14(11):1232-1238; the Danish Centre for Human Genome Research (http://biobase.dk/cgi-bin/celis), Celis J. E. et al., “Human 2-D PAGE databases for proteome analysis in health and disease: http://biobase.dk/cgi-bin/celis,” FEBS Lett. 1996 398(2-3):129-134, etc. Antibodies may be obtained from a variety of sources such as BD Transduction Laboratories (Lexington, Ky.) or Santa Cruz Biotechnology (Santa Cruz, Calif., USA).

Although, as discussed above, any of a broad class of ligands may be employed, for simplicity the embodiment is illustrated with reference to the use of antibody ligands. Immunological identification of the proteins on the membranes thus preferably involves the selection of antibodies having a high affinity and specificity for their targets. However, antibodies, both monoclonal or polyclonal, frequently recognize more then one protein in Western blotting detection. This cross-reactivity phenomenon becomes increasingly apparent as the concentration of antibody increases relative to that of the sample proteins. Hence, the first step in the antibody selection process preferably involves choosing antibodies (and their working concentrations) that consistently visualize preferably 1 but no more then 5 proteins on the same membrane. When the detector antibody binds to more than one spot, the undesired proteins (“false spots”) can be eliminated based on their X-Y positions on the membranes. Since the molecular weight and charge (pI) of a given protein is generally constant, it should appear at about the same coordinates on the gel each time it is run.

If two or more proteins in a sample are of similar size and charge—and therefore migrate to the same general vicinity on the gel—they would likely create overlapping spots if detected on the same membrane. In a preferred embodiment, the method of the present invention avoids this problem by designing the antibody cocktail to detect adjacent or overlapping proteins on different membranes.

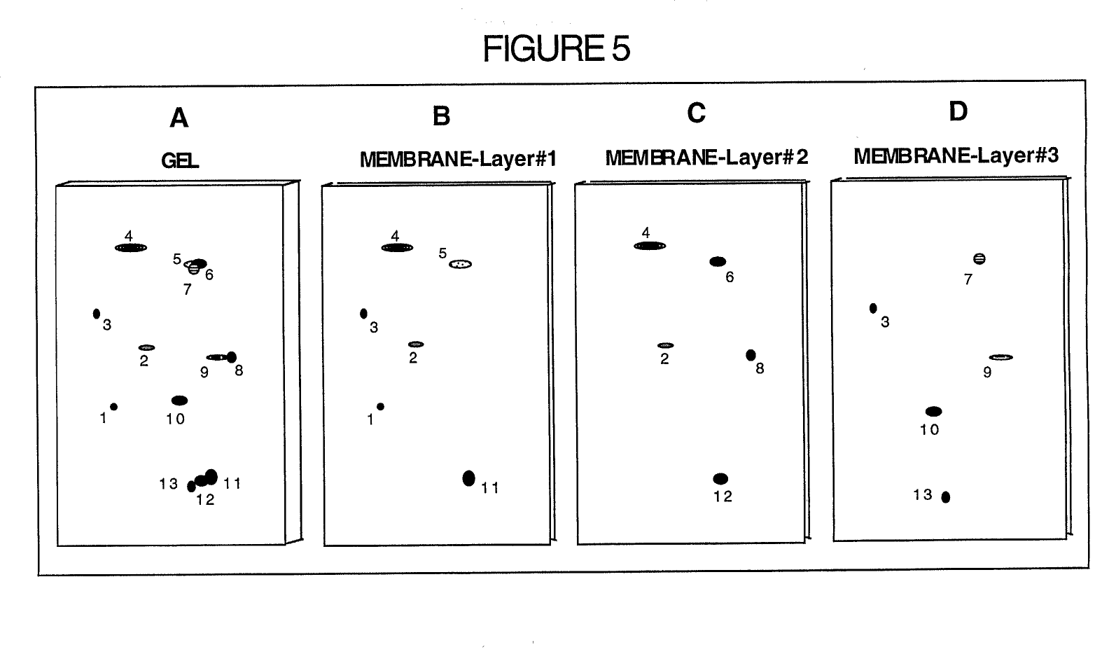

The cocktail design process can be readily understood with reference to the following hypothetical example (illustrated in FIG. 5). For simplicity in this example, thirteen proteins annotated as 1-13 in FIG. 5(a) are sought to be identified using only a three layer membrane stack. The ligands employed in the example are antibodies, and . three cocktails, one for each stack, each with 4-6 different antibodies, are employed.

For the first membrane cocktail (corresponding to layer one) antibodies are screened for protein spot 1 and the most specific antibody is selected. Antibodies for spots 2-5 are picked the same way. Because spots 6 and 7 overlap with spot 5 these are put aside for other layers. The second and third cocktails (corresponding to membrane layers two and three) are created using the same considerations: (1) if the spot position generated by any two antibodies cannot be easily distinguished, the antibodies will not be used in the same cocktail; (2) if any antibody results in a background spot near the spot generated by another antibody, the two antibodies will not be included in the same cocktail unless the background spot is remote from other spots on that layer (e.g. spots 2 and 4 on layer 2 created due to cross-reactivity from antibodies directed to other spots), in which case such cross-reactivity is simply ignored when the membrane spots are compared to the template. Applying these considerations to the hypothetical example results in three cocktails corresponding to the layers illustrated in FIGS. 5(b-c).

Once assembled, the antibody cocktails will be additionally tested for their specificity by two different control tests. In a first test, membranes made from the transfer of a single gel (or from several gels that contain the same sample and were prepared in the same manner) will be probed with cocktails that differ in only one antibody component (each cocktail will lack one of the antibodies). As a result of this procedure, immunoblotted membranes should differ from each other in only one spot. In a second test, antibody cocktail will be incubated for 0.5-12 hours at 4-25° C. with a mixture of epitopes (peptides or proteins) that are used for immunization. During this incubation, free antibodies bind to the appropriate epitopes and become immobilized and functionally inactive. Since the cocktail becomes depleted of free antibodies subsequent incubation of the membrane with this free antibody depleted mixture should yield no specific signal.

Each cocktail will also include one or more antibodies against “housekeeping” proteins (i.e., abundant structural proteins found in all eukaryotic cells such as actin, tubulin, etc.). Thus, for example, the antibodies employed with respect to membrane Layer#1 of FIG. 5 will contain an antibody to actin, which will result in the production of a spot These antibodies serve as internal landmarks to normalize samples for loading differences and to compensate for any distortion caused by gel running process. Once the cocktails are designed, they can be reused in any kit that seeks to identify the same panel of proteins that were identified in creating the cocktails, regardless of the origin of the sample.

In addition to identifying proteins of interest structurally, kits according to the present invention can also be employed to identify the functional state of proteins. One way to do so is to use phospho—specific antibodies to determine the phosphorylative state of protein(s) of interest. Another approach to identifying protein function is to first renature the proteins on the membranes by any of a number of techniques known in the art (such as incubating the membrane in Triton-X-100 ® (octylphenol ethylene oxide condensate). Once renatured, some proteins will regain their functional activity and one of several substrate degradation or modification assays known in art can be used. With this approach the activity of kinases, phosphates and metalloproteinases, etc., can be determined.

It should be appreciated that the present invention allows not only the simultaneous characterization of a large number of different proteins but also permits the characterization of a large number of characteristics of a single protein based on number of different characteristics. For example, the protein p70 S6 kinase, required for cell growth and cell cycle progression, is activated by phosphate group attachments (phosphorylation) to threonins on position 229 and/or 389 of the protein. Identification of this kinase according to the present invention would provide not only a determination of its presence or absence but also a demonstration of its activity. With kit 10, one can make four copies of the 2-D gel. The first membrane would be incubated in antibody specific for the whole protein to determine if this enzyme is present in the sample or not. The second membrane can be used in kinase assay to determine if the enzyme is active or not. The third membrane can be probed with phospho-p70 S6 kinase (Thr229) antibody to determine if activity of the enzyme is due to activation of this site. The fourth membrane can be probed with phospho-p70 S6 Kinase (Thr389) antibody to determine if the activity of the enzyme is due to activation of that site. And since all of these tests are done on the single sample (rather than different batches of the same sample) the information obtained is very reliable.

Antibody cocktails 14 are preferably stored in vials, preferably made of plastic or glass, and are combined in kit 10 to create a “panel” of protein targets of interests. Panels for scientific research may be grouped by the proteins involved in a particular cellular phenomenon such as apoptosis, cell cycle, signal transduction, etc. Panels for clinical diagnostics may be grouped by proteins associated with a particular disease such as Alzheimer's, prostate cancer, etc.

### Image Analysis Software

Software 20 is made available to users of kit 10 by providing it on a diskette to be included within kit 10 or by making it accessible for downloading over the Internet or a private intranet network, or by other means. The function of software 20 is to translate the visible spots generated by antibody cocktails 14 into useful information about the proteome of the sample being tested. This information primarily includes the quantity of the proteins in the test sample relative to a control and, in some cases, information about certain functional aspects of these proteins. Suitable software can be obtained from, or adapted from, any of a variety of sources (e.g., http://www.2dgels.com/home.html and http://expasy.proteome.org.au).

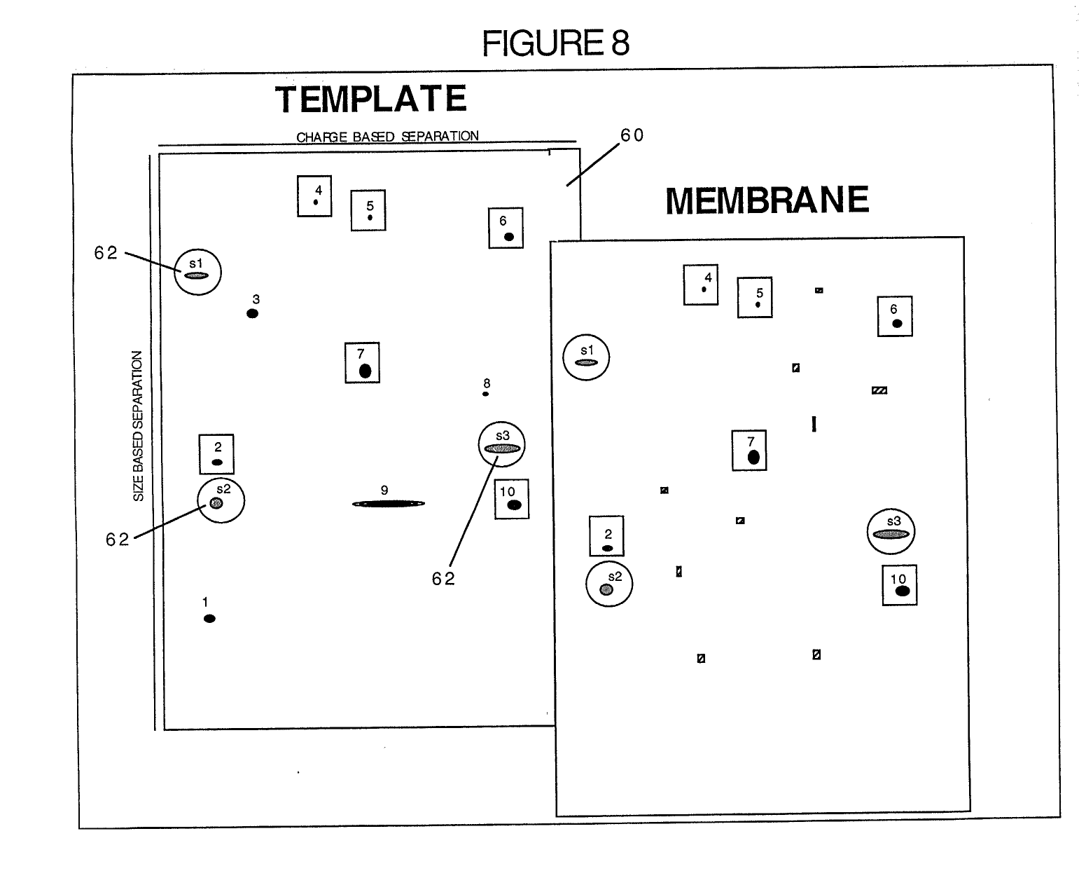

After it is determined which proteins will be identified on each layer for a given panel/kit a template image 60 is created corresponding to each layer (FIG. 8) and stored in software 20. The 2-D gel X-Y coordinates of each protein can be ascertained from any of a number of references and data bases (see above) Thus, referring to FIG. 8, template image 60 is the image of what a membrane would look like if all of the targeted proteins assigned to the layer are present in the sample being tested along with the landmark “housekeeping” proteins 62. Each antibody cocktail generates a unique dot pattern on the corresponding membrane to which it is applied as a result of the selection process outlined above. A template membrane 60 will be used by image processing software to analyze experimental membranes generated by users. Important feature of the template is existence of the internal landmarks 62. These spots will originate from the set of antibodies targeted against housekeeping proteins present in every sample regardless of origin. Since their relationship always stay the same these landmarks will serve to normalize samples for loading differences and to compensate for any distortion caused by gel running process.

Image analysis will start with digitalized image of the experimental membranes. As the first step user will have to match templates with the membranes. Software will then compare image of the template and image of the membrane and perform alignment of spots. User will have an option of visual alignment control and ability to hand correct any manor discrepancies. The second step of analysis will include densitometric readings of the spots on experimental membranes. This data will be stored in the database. The third step will include numerical data manipulation. Intensity value of each experimental spot on the membrane will be divided with values of the landmark spots. This step will generate normalized intensity values for each spot on the membrane. All the spots of interest can thus be compared with each other.

Software 20 preferably allows the user to select the kind of comparative analysis to be performed (i.e. comparing the spots present in one sample with those in another sample or comparing the spots present on one membrane with those of another membrane within the same membrane stack). Results of the analysis is displayed in tabular format and user is given the option to go back and compare magnified sections of the images of interest.

### Uses and Applications

With reference to FIG. 1, kit 10 may be used to identify proteins that have been separated on electrophoresis gels, both two dimensional gels 42 and one dimensional gels (not shown).

Proteins are isolated from a biological sample and separated on the gel 42 according to techniques well known in the art such as those described in Manabe, T. “Combination of electrophoretic techniques for comprehensive analysis of complex protein systems,” Electrophoresis. 2000 21(6):1116-22; Oh, J. M. et al., Mining protein data from two-dimensional gels: tools for systematic post-planned analyses,” Electrophoresis. 1999 20(4-5):766-774; Dunn, M. J. “Two-dimensional gel electrophoresis of proteins,” J Chromatogr. 1987 418:145-185;

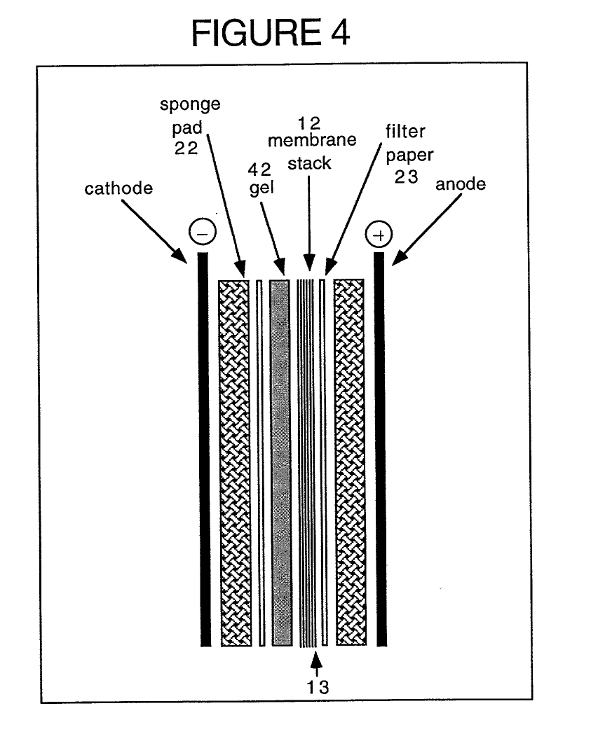

After gel 42 is run, it is removed from the electrophoresis apparatus and sandwiched and placed in a transfer apparatus such as the type typically used in creating Western blots. Such devices are available , for example, from Biorad Laboratories, Inc., Novex, Inc. and Amersham Pharmaceia. Membrane stack 12 is positioned between the electrodes adjacent to gel 42 as illustrated in FIG. 4. While only about a half-dozen membranes are shown in FIG. 4 it should be appreciated that up to one hundred may be employed depending on the number of targets sought to be identified in a panel, the quantity of proteins present in the sample, and the thickness of the material employed to construct membranes 13. Optionally, membranes 13 may be packaged in a suitable sealed enclosure or frame (not shown) to maintain their integrity and prevent contamination. Sponge pads 22, preferably constructed of foam, rubber or filter paper and layers of filter paper 23 are also sandwiched between the electrodes as shown in FIG. 4. Transfer buffer (25 mM Tris pH 8.3, 192 mM glycine, 0.025% SDS and 20% methanol) is applied to elute and transfer proteins from the gel 42 to the membranes 13. Any of a variety of conventional methods for effecting such transfer may be employed, including wet tank transfer, and semi-dry transfer. In a wet tank transfer, the membranes are immersed into a tank containing buffer; in a semi-dry transfer, the membranes are blotted with moist pads. In both cases, the membranes are subjected to a voltage potential (e.g., 125-150 mAmps for 1-10 hours). In such transfer, it is important that a tight contact be created between the membranes and the gel. Wet tank transfer is preferred. For a membrane of 10×10 cm², a tank containing 400-500 ml of buffer may be employed. Preferred transfer conditions are 60-110 mAmps for 1-2 hours. After transfer the membranes are separated and incubated with the detector antibody. Antibodies are selected based on the types of targets sought. Membranes are washed in a buffer, and the protein/detector complex can be visualized using a number of techniques such as ECL, direct fluorescence, or calorimetric reactions. ECL is preferred. Commercially available flatbed scanners may be employed in conjunction with film. Alternatively, specialized imaging instrumentation for ECL, such as the Kodak IMAGE STATION available from NEN may be utilized and digital imaging software can be employed to display the images according to the preference of the user, as discussed above.

Kit 10 may be used to identify proteins in any biological sample including bodily fluids (e.g. blood, plasma, serum, urine, bile, cerebrospinal fluid, aqueous or vitreous humor, or any bodily secretion), a transudate, an exudate (e.g. fluid obtained from an abscess or any other site of infection or inflammation), or fluid obtained from a joint. Additionally, a biological sample can be obtained from any organ or tissue (including or autopsy specimen) or may comprise cells, or extracts thereof.

In addition to use with 2-D gels, as described throughout this specification, the present invention may be employed to identify proteins that have been separated by a 1-D gel such as conventional gels for separating proteins by size, and gel shift assays. Gel shift assays (also known as “mobility shift assays”) are the most commonly used tool for studying protein—DNA interactions. The assay is based on labeling of the DNA fragment that contains presumptive protein binding site and incubation of that labeled fragment with protein that binds to that site. If they interact, complex will be formed. If source of protein is a cell extract (rather than a solution of in vitro synthesized proteins) the complex may contain number of proteins, of unknown identity, that interact with each other. After binding, a mixture of DNA and proteins is loaded onto a non-denaturing polyacrylamide gel and the proteins are separated based on their size. DNA-protein complexes are visualized by exposure to X-ray film, or by other means. The higher the bands are in the gel, the larger the size of the DNA-protein complex. In most cases, this type of analysis does not reveal identity of the protein(s) in the complex.

It should be appreciated that because the size of the membrane array can be varied, the user has the option of analyzing a large number of different samples in parallel, thereby permitting direct comparison between different samples (e.g., different patient samples, or patient samples and a reference standard, or samples of different tissues or species, etc.). For example, different samples from the same patient at different stages of disease can be compared in a side-by-side arrangement as can samples from different patients with the same disease.

Having now described the invention in detail, the same may be better understood and its numerous objectives and advantages become more apparent to those familiar in the art by reference to the following Examples which are not intended to restrict or limit the subject matter of the invention.

### EXAMPLE 1

### Transfer And Capture Of Proteins From A 1-D Gel

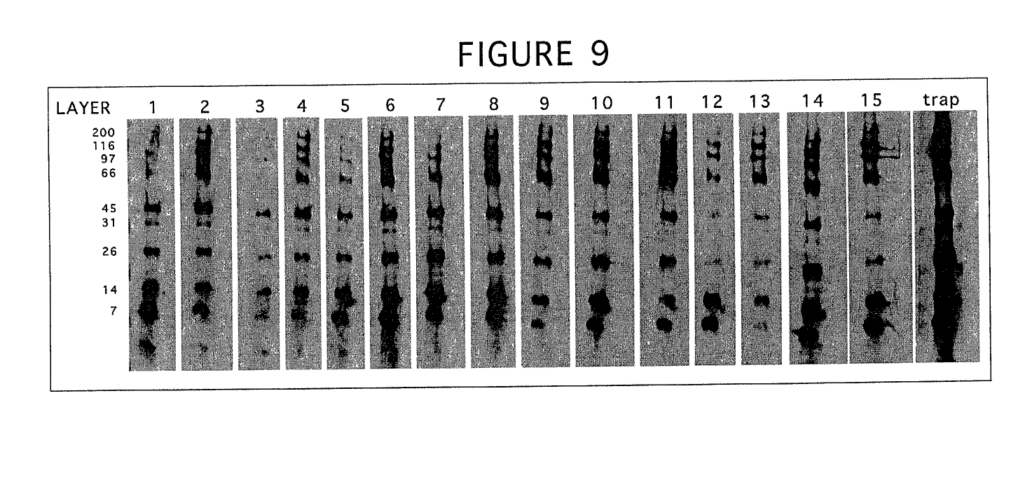

This experiment demonstrates that PCNC membranes, with their high binding affinity but low capacity for the proteins eluted from the gel, can be used to make multiple copies of a gel. 1.0 μg/lane of biotinylated protein marker (Vector Laboratories, Inc) was separated by 15% PAGE and electro-transferred in 25 mM Tris, 192 mM glycine, 0.025% SDS and 20% methanol (60-110 V for 1-2 hours) through a stack of polycarbonate coated nitrocellulose (PCNC) membranes (as described in U.S. patent application Ser. No. 09/718,990, herein incorporated by reference; the number of membranes per stack was 5-20, depending on the experiment. At the end of the stack, one pure nitrocellulose membrane was used to capture all of the proteins that were not bound to PCNC layers (NC trap). Transfer was performed from 0.5-3 hours on 60-110 V in a Ready Gel Cell apparatus (BioRad). After transfer, membranes were rinsed in 50 mM Tris pH 8.0 and 150 mM NaCl (TBST buffer), blocked, for 10-60 minutes in 1× casein solution (Vector Laboratories, Inc.) and incubated for 30 minutes in VECTASTAIN ABC-AmP reagent (Vector Laboratories, Inc.). Membranes were washed again in TBST, rinsed in 0.1 M TRIS pH 9.5, incubated in DuoLux reagent (Vector Laboratories, Inc.) for 3-5 minutes and exposed to Biomax MR film (Kodak). An example of one representative experiment is shown in FIG. 9.

The results demonstrated that:

1. PCNC stack of membranes did not interfere with Western blotting procedure—proteins were transferred from the gel to the NC trap;

2. Wide range of protein sizes were transferred through the stack with very similar transfer efficiency—7 kDa-200 kDa proteins were detected on the NC trap; and

3. PCNC layers captured proteins regardless of their size.

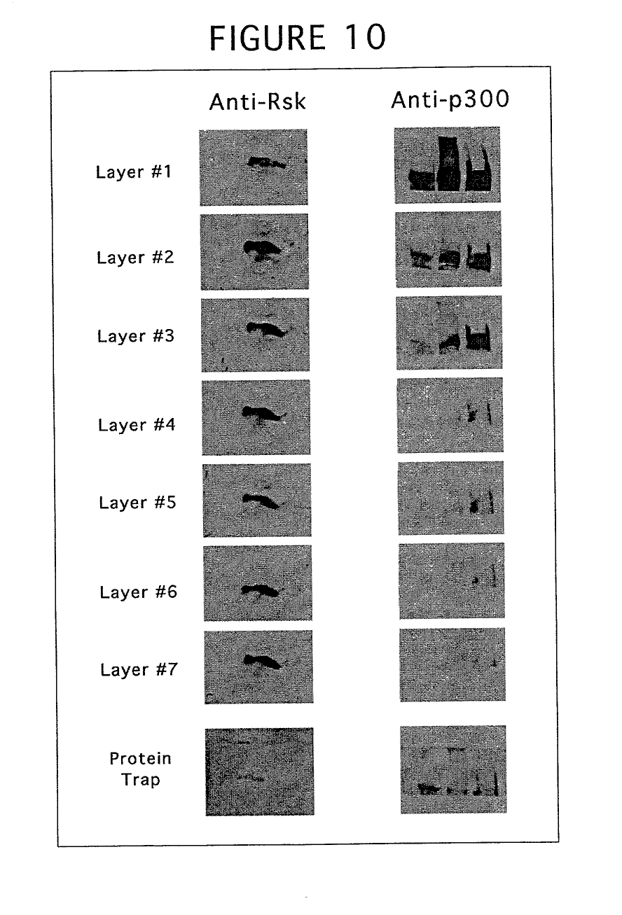

In order to determine compatibility of PCNC membranes with immunodetection, Jurkat cell were lysed in 50 mM TRIS pH 8.0 and 1% SDS and total of 20 μg/lane of protein was separated by 15% PAGE and electro-transferred in 25 mM TRIS,192 mM glycine, 0.025% SDS and 20% methanol (60-110 V for 1-2 hours) through a stack of PCNC membranes. All of the membranes were incubated in primary anti-Rsk (1:100, Transduction Laboratories) and anti-p300 (1:500, Transduction Laboratories) antibody, washed in TBST buffer, incubated with the complex of secondary antibody and alkaline phosphatase, and washed again. The location of the protein was visualized by ECL (DuoLux, Vector Laboratories, Inc.) and Biomax MR film (Kodak). The results, shown in FIG. 10, demonstrated that PCNC membranes are very suitable for this type of protein detection. Each membrane captured just enough of a protein to be detected by immunological methods but single membrane did not capture too much so number of copies of the same gel were made.

### EXAMPLE 2

### Transfer And Capture Of The Proteins From A 2-D Gel

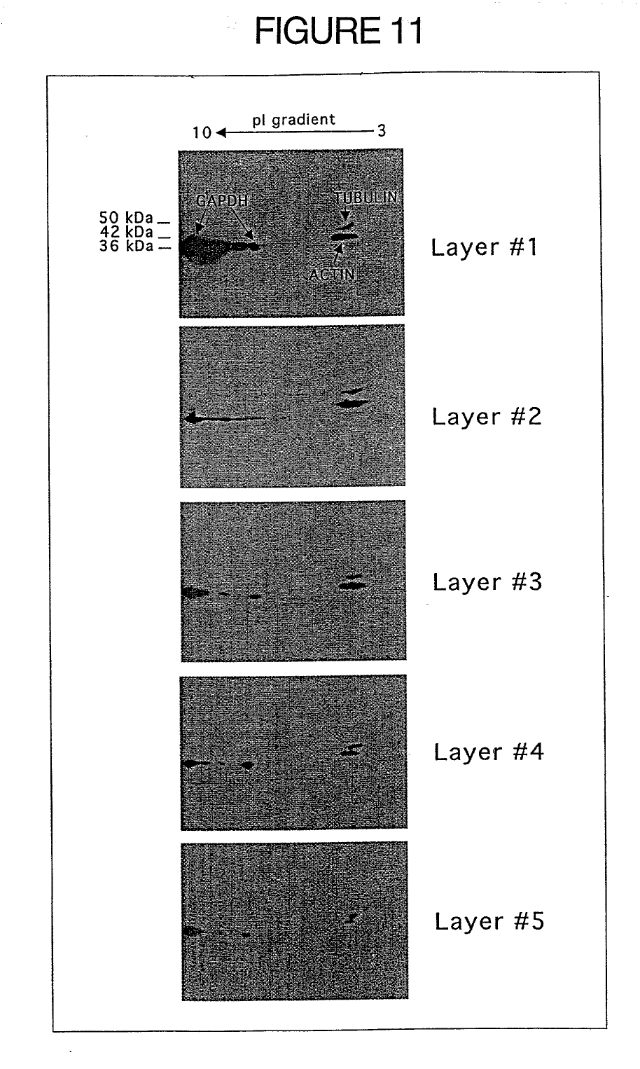

2-D protein gels have greater separation capabilities than 1-D gels. Two dimensional separation allows identification of hundreds or even thousands of proteins on the same gel. Proteins separated by 2-D gels are identified by protein sequencing or immunological features. Sequencing requires expensive equipment and highly trained operators and is limited to a small number of privileged groups. Immunodetection is easier to do but it is of low throughput since traditional blotting procedures generate only one membrane copy of gel. As described above, one can make at least 10 and possibly even larger number of 1-D gel copies using PCNC membranes. In order to find out if 2-D gel can be “copied” the same way, the proteins present in 500 μg of Jurkat cell protein lysate were separated on 2-D PAGE. A commercial immobilized pH gradient (IPG) from 3.0 to 10.0 was used for first-dimension separation (Pharmacia Biotech, Uppsala, Sweden). Eight to 12 hours in-gel sample rehydration was used for protein loading. Proteins were separated for final of 15,000-30,000 Vhrs. After equilibration, the IPG gel strips were transferred onto vertical gradient gel (4-20%, Novex) for second dimension separation. After electrophoresis, the gel was transferred into 25 mM Tris,192 mM glycine, 0.025% SDS and 20% methanol (60-110 V for 1-2 hours) through a stack of 5 PCNC membranes. After such transfer, the membranes were rinsed in TBST buffer, blocked for 10-60 minutes in 1× casein solution (Vector Laboratories, Inc.) and incubated overnight at 4° C. in anti-GAPDH (1:5,000, Chemicon), anti-beta-actin (1:5,000, Sigma) and anti alpha-tubuli (1:1,000, Calbiochem) antibody, washed in TBST, incubated in the complex of secondary antibody and alkaline phosphatase, and washed again. The location of the protein was visualized by ECL (DuoLux, Vector Laboratories, Inc.) and Biomax MR film (Kodak). In this experiment, antibodies were first applied separately to 3 different membranes (from 3 different gels) to find exact spatial location of each protein in the 2-D gel. These 3 proteins differ in their size and charge and were spatially separated from each other on the gel (not shown). In order to increase the throughput of immuno-detection, all three antibodies were mixed together and applied as a cocktail to all 5 membranes from the same gel. The results of this experiment are shown in FIG. 11. The Results demonstrate that by generating at least 5 replicas of the same gel and by using the antibody cocktail approach. of the present invention increased throughput of the immunological protein identification on 2-D gels was obtained.

### EXAMPLE 3

### Use Of Layered Membranes For Protein-DNA Complexes Identification

The following experiment was conducted to demonstrate the ability of the layered membranes of the present invention to speed up and simplify the identification of the proteins of a protein-DNA complex. This goal was achieved by making copies of the gel and immuno-probing each of the membranes with a different antibody of interest.

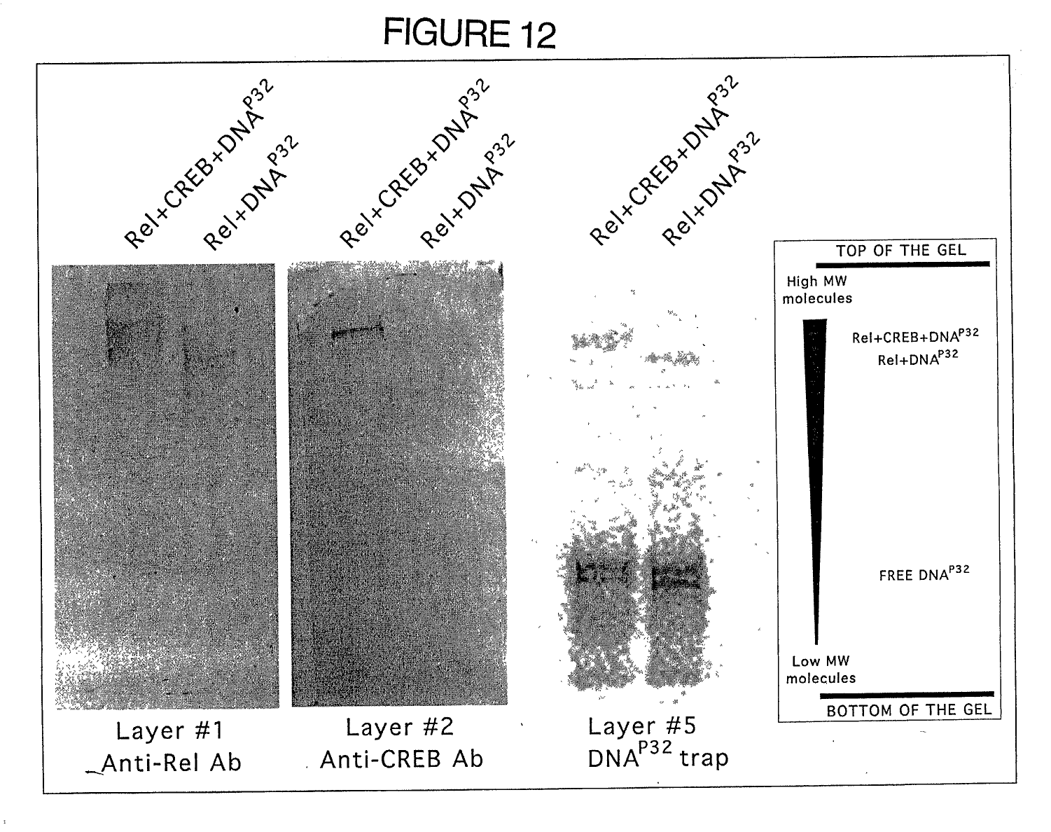

250 ng of recombinant his6-c-rel and 120 ng of purified recombinant his6-CREB were incubated alone or in combination with 0.2 ng of 32P 5′ labeled duplex oligonucleotide encoding the sequence 5′ TCGACCTCTTCTGATGACTCTTTGGAATTTCTTTAAACCCCCA 3′ (SEQ ID NO.:1), in 10 μl of buffer containing 10 mM Hepes, 50 mM NaCl, 20% glycerol, 4 mM BME. The reactions was allowed to proceed at room temperature for 30 min. Samples were then separated by electrophoresis on 4% polyacrylamide gel at 180 Volts for 1 hour, transferred in 25 mM TRIS,192 mM glycine, 0.025% SDS and 20% methanol (60-110 V for 1-2 hours) through a stack made of 4 PCNC membranes and 1 NA45 DEAE (Schleicher & Schuell) membrane. This last layer of charged cellulose was used to trap all the DNA released from the gel. After transfer, registration marks were made by 19G needle and DEAE membrane was dried down and exposed overnight to phosphoimager screen and visualized on Phosphorimager: SI (Molecular Dynamics). First and second PCNC membranes were rinsed in TBST buffer, blocked for 10-60 minutes in 1× casein solution (Vector Laboratories, Inc.) and incubated overnight at 4° C. in anti-ral antibody (1:200, NCI Laboratory of Pathology, Transcription Regulation Unit Chief, Dr. Kevin Gardner) and anti-His (1:10,000, Stratagene), washed in TBST, incubated in the complex of secondary antibody and alkaline phosphatase, washed again and location of the protein was visualized by ECL (DuoLux, Vector Laboratories, Inc.) and Biomax MR film (Kodak). Images of all of the membranes were aligned in Adobe Photoshop (FIG. 12). The results demonstrated that the layered membrane array of the present invention provides fast and reliable identification of proteins from a protein complex.

Although certain presently preferred embodiments of the invention have been described herein, it will be apparent to those skilled in the art to which the invention pertains that variations and modifications of the described embodiment may be made without departing from the spirit and scope of the invention. Accordingly, it is intended that the invention be limited only to the extent required by the appended claims and the applicable rules of law. The references cited above are hereby incorporated herein in their entirety.

What is claimed is:

1. A method for analyzing the proteome of a biological sample comprising the steps of:
(a) separating said protein from another protein present in said sample;
(b) transferring a portion of said separated protein to a plurality of membranes in a stacked configuration;
(c) incubating each of said membranes in the presence of one or more species of predetermined ligand molecules under conditions sufficient to permit binding between said separated protein and a ligand capable of binding to such protein; and
(d) analyzing said proteome by determining the occurrence of binding between said protein and any of said species of predetermined ligand molecules.

2. The method of claim 1, wherein said separation of said protein from another protein present in said sample is accomplished by electrophoresis.

3. The method of claim 2, wherein said electrophoresis is 2-dimensional gel electrophoresis.

4. The method of claim 1, wherein said sample is obtained from mammalian cells or tissue.

5. The method of claim 4, wherein said mammal is a human.

6. The method of claim 1, wherein said transferring of a portion of said separated protein is accomplished by gel transfer.

7. The method of claim 1, wherein said mammalian cells or tissue are human cells or tissue.

8. The method of claim 1, wherein said separated protein is a product of a human gene.

9. The method of claim 1, wherein at least one of said species of ligand is selected from the group consisting of an antibody, an antibody fragment, a single chain antibody, a receptor protein, a solubilized receptor derivative, a receptor ligands, a metal ion, a virus, a viral protein, an enzyme substrate, a toxin, a toxin candidate, a pharmacological agent, and a pharmacological agent candidate.

10. The method of claim 9, wherein at least one of said species of ligand is an antibody or an antibody fragment.

11. The method of claim 9, wherein at least one of said species of ligand is a receptor protein, a solubilized receptor derivative, or a receptor ligand.

12. The method of claim 9, wherein at least one of said species of ligand is a pharmacological agent or pharmacological agent candidate.

13. The method of claim 9, wherein the binding of at least one of said species of ligand is dependent upon the structure of said separated protein.

14. The method of claim 9, wherein the binding of at least one of said species of ligand is dependent upon the biological function of said separated protein.

15. The method of claim 1, wherein at least one of said membranes is incubated with more than one species of ligand.

16. The method of claim 1, wherein at least 2 membranes are employed.

17. The method of claim 16, wherein at least 10 membranes are employed.

18. The method of claim 16, wherein at least 20 membranes are employed.

19. The method of claim 1, wherein at least 2 ligand species are employed.

20. The method of claim 19, wherein at least 10 ligand species are employed.

21. The method of claim 19, wherein at least 20 ligand species are employed.

22. The method of claim 1, wherein said step (c) is performed before said step (a).

23. A method for uniquely visualizing a desired predetermined protein if present in a biological sample, comprising the steps:
(a) separating the proteins present in said sample from one another;
(b) transferring a portion of the separated proteins of said sample to a plurality of membranes in a stacked configuration;
(c) incubating each of said membranes in the presence of one or more species of predetermined ligand molecules under conditions sufficient to permit binding between desired predetermined protein and a ligand capable of binding to such protein; and
(d) visualizing any binding between said protein and any of said species of predetermined ligand molecules.

24. A kit for analyzing a proteome comprising:
(a) a plurality of membranes, each having a specific affinity for at least one protein, and
(b) a plurality of reagent species, each adapted to detect one or more specific proteins bound to said membranes.

25. The kit of claim 24, which additionally contains instructions setting forth the particular groups of reagents to be applied to each of said membranes.

26. The kit of claim 24, wherein said membranes comprise a porous substrate having a thickness of less than about 30 microns.

27. The kit of claim 26, wherein said membranes are polycabonate membranes, coated with a material for increasing the affinity of the membrane to biomolecules.

28. The kit of claim 27, wherein said membranes are coated with nitrocellulose.

29. The kit according to claim 24 wherein said reagent species are selected from the group consisting of an antibody, an antibody fragment, a single chain antibody, a receptor protein, a solubilized receptor derivative, a receptor ligands, a metal ion, a virus, a viral protein, an enzyme substrate, a toxin, a toxin candidate, a pharmacological agent, and a pharmacological agent candidate.

30. A kit for uniquely visualizing a desired predetermined protein if present in a biological sample, comprising:
(a) a plurality of membranes, each having a specific affinity for at least one protein, and
(b) a plurality of reagent species, each adapted to detect said desired predetermined protein if bound to said membranes.


SEQUENCE LISTING (1 sequence)

SEQ ID NO: 1 - DNA, 43 bp, Artificial Sequence
  Description of Artificial SequenceBinds His6-c-rel and his6-CREB
tcgacctcttctgatgactctttggaatttctttaaaccccca